(19) United States
(12) Patent Application Publication
(10) Pub. No.: US 2019/0247069 A1
(43) Pub. Date: Aug. 15, 2019

(54) ULTRASONIC PROBE

(71) Applicant: OLYMPUS CORPORATION, Tokyo (JP)

(72) Inventors: Ken FUJISAKI, Sagamihara-shi (JP); Takamitsu SAKAMOTO, Hachioji-shi (JP); Michio TAKAYAMA, Tokyo (JP); Hideto YOSHIMINE, Hachioji-shi (JP)

(73) Assignee: OLYMPUS CORPORATION, Tokyo (JP)

(21) Appl. No.: 16/394,487

(22) Filed: Apr. 25, 2019

Related U.S. Application Data
(63) Continuation of application No. PCT/JP2016/082176, filed on Oct. 28, 2016.

(51) Int. Cl. A61B 17/32

(52) U.S. Cl. CPC A61B 17/32002; A61B 17/320068; A61B 2017/32007; A61B 2017/320052; A61B 2017/320084

(57) ABSTRACT

An ultrasonic probe can include a probe main body that receives ultrasonic vibration generated by an ultrasonic transducer and a treatment unit disposed on a distal end of the probe main body. The treatment unit can include a resection portion and a guide unit that each have a projected shape when viewed along a longitudinal axis of the ultrasonic probe. The resection portion can resect a bone that needs to be treated along a resection direction. The guide unit can protrude from the resection portion on a distal end of the treatment unit. The guide unit can also help to maintain the resection direction relative to a central axis of the hole.

### CROSS-REFERENCE TO RELATED APPLICATION

This application is a continuation of International Application No. PCT/JP2016/082176, filed on Oct. 28, 2016, the entire contents of which are incorporated herein by reference.

### BACKGROUND

The present disclosure relates to an ultrasonic probe.

An ultrasonic probe can have a treatment unit provided on its distal end. The ultrasonic probe can transmit ultrasonic vibration to the treatment unit so that when the treatment unit contacts a bone, the treatment unit can form a concave hole in the bone.

Ultrasonic probes can be used in, for example, anterior cruciate ligament (ACL) reconstruction at a knee joint. In the ACL reconstruction, a small hole (through hole) for inserting a fixator that fixes a constructed tendon transplant is made in a femur and/or a tibia by a drill or the like. Thereafter, by moving the treatment unit closer to the bone along the small hole, a large hole (concave hole) for inserting the constructed tendon transplant is made around the small hole, or at a position including the small hole. The tendon transplant is constructed such that a transverse section has an approximately rectangular shape or a shape similar to a rectangular shape. Therefore, a cross-sectional shape of the concave hole made in the bone corresponds to a cross-sectional shape of the tendon transplant, i.e., corresponds to a polygonal hole, such as a rectangular hole, or an elliptical hole, rather than a circular hole. In this case, a lot of skill is needed to match a central axis of the large hole (concave hole) made by the ultrasonic probe with a central axis of the small hole that is a circular hole.

### SUMMARY

An ultrasonic probe can be provided with a probe main body that receives ultrasonic vibration generated by an ultrasonic transducer. The ultrasonic probe can also include a treatment unit that is disposed on a distal end side of the probe main body along a longitudinal axis. The treatment unit can make a hole in a bone that needs to be treated, and the treatment unit can include a resection portion and a guide unit that protrudes from a distal end of the resection portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a treatment system according to an exemplary embodiment;

FIG. 2 is a schematic diagram illustrating a configuration of an ultrasonic treatment tool according to an exemplary embodiment;

FIG. 3 is a perspective view schematically illustrating a configuration of a distal end portion of an ultrasonic probe according to an exemplary embodiment;

FIG. 4 is a side view schematically illustrating a configuration of the distal end portion of the ultrasonic probe according to an exemplary embodiment;

FIG. 5A is a schematic diagram illustrating a projected shape of a distal end portion of an ultrasonic probe according to an exemplary embodiment viewed from a distal end side to a proximal end side along a longitudinal axis;

FIG. 5B is a schematic diagram illustrating a projected shape of a distal end portion of an ultrasonic probe according to an exemplary embodiment viewed from a distal end side to a proximal end side along a longitudinal axis;

FIG. 6A is a diagram schematically illustrating a cross-section of a bone in which a small hole is made, where the cross-section includes a central axis of the small hole;

FIG. 6B is a diagram schematically illustrating the bone in which the small hole is made, when viewed from a front side;

FIG. 7A is a diagram schematically illustrating a state in which the distal end portion of the ultrasonic probe according to an exemplary embodiment is inserted in the small hole made in the bone;

FIG. 7B is a diagram schematically illustrating a state in which the distal end portion of the ultrasonic probe according to an exemplary embodiment makes a large hole in the bone;

FIG. 8A is a diagram schematically illustrating a cross-section of the bone in which the small hole and the large hole are made, where the cross-section includes the central axis of the small hole;

FIG. 8B is a diagram schematically illustrating the bone in which the small hole and a concave hole (large hole) with a polygonal cross-section are made, when viewed from the front side;

FIG. 8C is a diagram schematically illustrating the bone in which the small hole and a concave hole (large hole) with an elliptical cross-section are made, when viewed from the front side;

FIG. 9A is a perspective view schematically illustrating a configuration of a distal end portion of an ultrasonic probe according to an exemplary embodiment;

FIG. 9B is a diagram schematically illustrating a state in which a concave hole (large hole) is made in a bone while the distal end portion of the ultrasonic probe according to an exemplary embodiment is inserted in a small hole; and

FIG. 10 is a perspective view schematically illustrating a distal end portion of an ultrasonic probe according to a modification of an exemplary embodiment.

### DETAILED DESCRIPTION

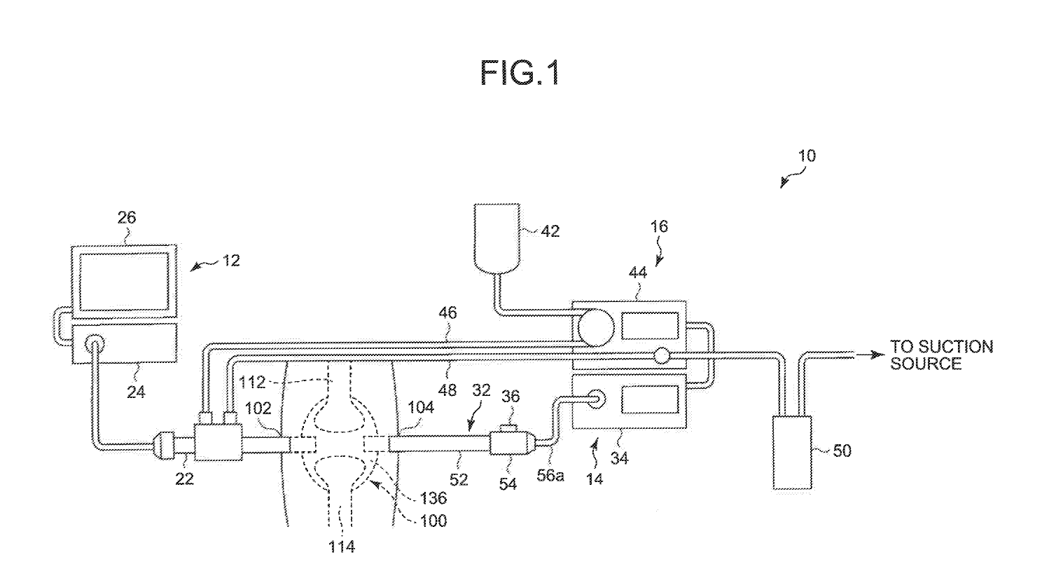

FIG. 1 is a diagram illustrating a treatment system 10 that is used for treating a knee joint 100. The treatment system 10 includes an arthroscope device 12, a treatment device 14, and a perfusion device 16.

The arthroscope device 12 includes an arthroscope 22 that observes a joint space 136 inside the knee joint 100 of a patient, an arthroscope controller 24 that performs image processing based on a subject image captured by the arthroscope 22, and a monitor 26 that produces an image generated by the arthroscope controller 24 through the image processing. The arthroscope 22 is inserted into the joint space 136 of the knee joint 100 from a first portal 102 by which the inside of the knee joint 100 and the outside of the skin of the patient are communicated.

The treatment device 14 includes a treatment unit 32, a controller 34, and a switch 36. The switch 36 is illustrated as a hand switch in FIG. 1, but may be a foot switch. The controller 34 supplies electrical energy for generating ultrasonic vibration to the treatment unit 32, in accordance with operation on the switch 36. The treatment unit 32 is inserted into the joint space 136 of the knee joint 100 from a second portal 104 by which the inside of the knee joint 100 and the outside of the skin of the patient are communicated.

The perfusion device 16 includes a liquid source 42 for storing perfusion solution, such as saline solution, a perfusion pump unit 44, and a suction bottle 50. One end of a solution sending tube 46 is connected to the liquid source 42. The other end of the solution sending tube 46 serving as a solution sending passage is connected to the arthroscope 22. Therefore, the perfusion pump unit 44 is able to send the perfusion solution from the liquid source 42 to the inside of the joint space 136 of the knee joint 100 via the arthroscope 22. One end of a solution discharging tube 48 is connected to the suction bottle 50. The other end of the solution discharging tube 48 serving as a solution discharging passage is connected to the arthroscope 22.

Therefore, the perfusion pump unit 44 is able to discharge the perfusion solution from the inside of the joint space 136 of the knee joint 100 to the suction bottle 50 via the arthroscope 22.

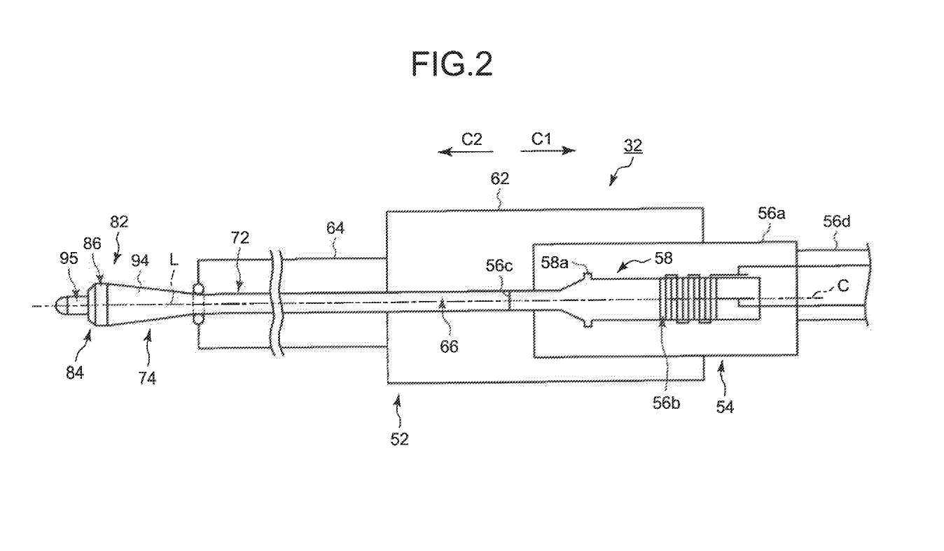

FIG. 2 is a diagram illustrating a configuration of the treatment unit 32. A central axis C is defined as illustrated in FIG. 2. Here, a direction along the central axis C is referred to as a longitudinal direction. One end side in the longitudinal direction is referred to as a distal end side (an arrow C2 side in FIG. 2), and a side opposite to the distal end side is referred to as a proximal end side (an arrow C1 side in FIG. 2).

The treatment unit 32 includes an ultrasonic treatment tool 52 and an ultrasonic transducer unit 54. It is preferable that the ultrasonic transducer unit 54 is removably attached to the ultrasonic treatment tool 52, but the ultrasonic transducer unit 54 may be integrated with the ultrasonic treatment tool 52.

The ultrasonic transducer unit 54 includes a housing (transducer case) 56a. The housing 56a is provided inside with a bolt-clamped Langevin-type transducer 56b including a piezoelectric element that converts supplied electrical energy into ultrasonic vibration. One end of a cable 56d is connected to the transducer (ultrasonic transducer) 56b. The other end of the cable 56d is connected to the controller 34. By supplying electric current (alternating current) to the transducer (ultrasonic transducer) 56b from the controller 34 via the cable 56d, the transducer 56b generates ultrasonic vibration, which then allows the transducer 56b to resonate at a predetermined frequency. An ultrasonic probe 66 (to be described later) is attached to a distal end of the transducer 56b.

The ultrasonic treatment tool 52 includes a housing (handle) 62, a tubular body (external tube) 64 that extends from the housing 62 along the central axis C, and the ultrasonic probe 66 inserted in the tubular body 64. The tubular body 64 is attached to the housing 62 from the distal end side. The housing 62 and the tubular body 64 are made of a material having electrical insulation property. The housing 56a of the ultrasonic transducer unit 54 is removably attached to the housing 62 of the ultrasonic treatment tool 52.

The ultrasonic probe 66 extends from the distal end side to the proximal end side. The ultrasonic probe 66 is made of a material, such as titanium alloy, that has high vibration transmissibility. A proximal end of the ultrasonic probe 66 is connected to a connecting portion 56c of the ultrasonic transducer unit 54. Ultrasonic vibration generated by the transducer 56b is transmitted to a distal end of the ultrasonic probe 66 via the connecting portion 56c. In this case, longitudinal vibration occurs in the ultrasonic probe 66 in a direction parallel to the central axis C due to the ultrasonic vibration. In other words, the ultrasonic probe 66 is a vibration transmitting member capable of transmitting the ultrasonic vibration from the proximal end side to the distal end side.

Meanwhile, a rotation knob (not illustrated) that is a rotation operating member may be attached to the housing 62 of the ultrasonic treatment tool 52. The rotation knob is rotatable about a central axis of the tubular body 64 relative to the housing 62. By rotating the rotation knob, the housing 56a of the ultrasonic transducer unit 54, the tubular body 64, and the ultrasonic probe 66 rotate together about the central axis C relative to the housing 62.

The ultrasonic probe 66 includes a probe main body 72 and a treatment unit 74 that is disposed on a distal end side of the probe main body 72. The probe main body 72 extends along the central axis C. The treatment unit 74 extends from a distal end of the tubular body 64 to the distal end side. In other words, in the ultrasonic probe 66, the treatment unit 74 is formed by a portion protruding from the tubular body 64. The treatment unit 74 comes in contact with a bone, which is a treatment target, while receiving the ultrasonic vibration, to thereby resect the bone in the contact portion and make a hole in the bone.

It is preferable that the probe main body 72 is constructed in a straight manner. Here, a longitudinal axis L of the treatment unit 74 is defined. The treatment unit 74 may be extended from a distal end of the probe main body 72 to the distal end side in a straight manner or in an appropriately bent manner.

Therefore, the central axis C of the probe main body 72 and the longitudinal axis L of the treatment unit 74 may coincide with each other or may deviate from each other. In this example, it is assumed that the longitudinal axis L coincides with the central axis C.

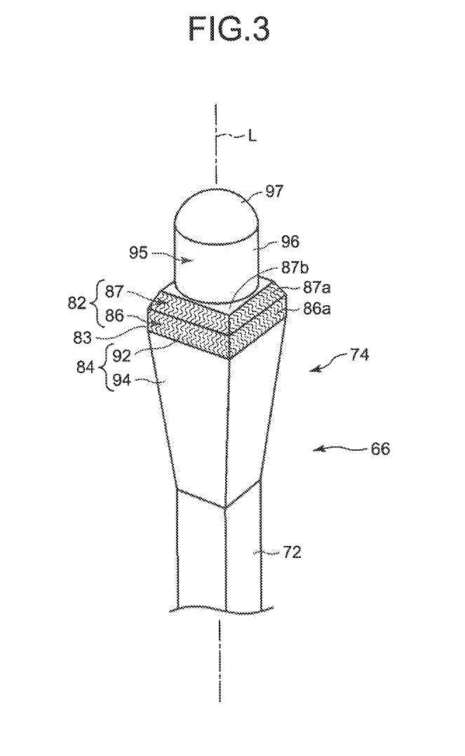

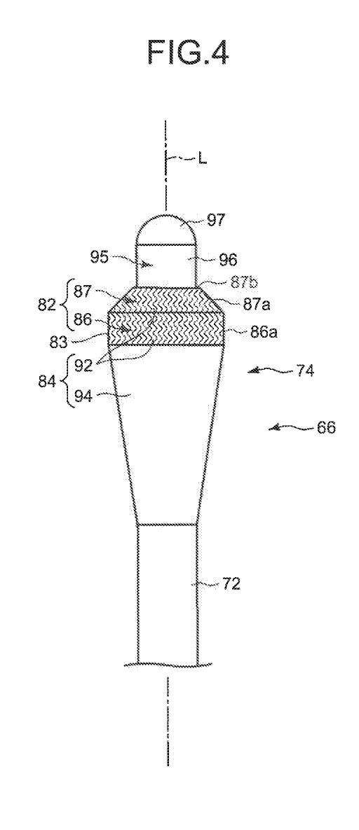

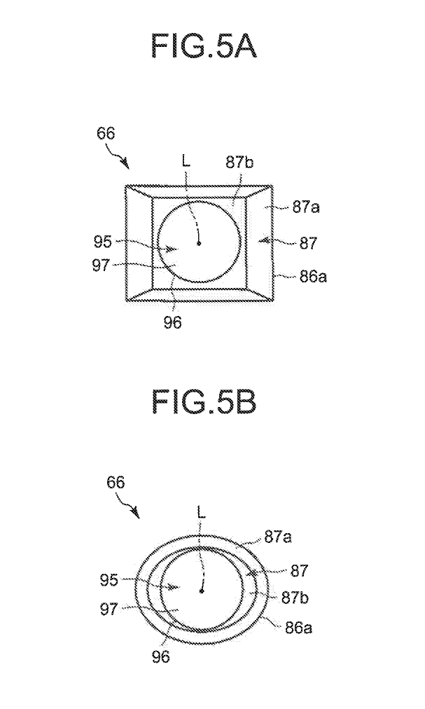

With reference to FIG. 3 to FIG. 5B, a configuration of the treatment unit 74 will be described. FIG. 3 is a perspective view illustrating the configuration of the treatment unit 74. FIG. 4 is a diagram illustrating the treatment unit 74 viewed from one direction perpendicular to the longitudinal axis L. FIG. 5A and FIG. 5B are diagrams illustrating projected shapes of the treatment unit 74 viewed from the distal end side to the proximal end side along the longitudinal axis L.

As illustrated in FIG. 3 to FIG. 5B, the treatment unit 74 includes side surfaces 83. The side surfaces 83 form an outer peripheral surface of the treatment unit 74. The treatment unit 74 includes a resection portion 82. It is preferable that a projected shape of the resection portion 82 is a polygon, such as an approximate rectangle, or an ellipse. In some embodiments, the projected shape of the resection portion 82 viewed from the distal end side to the proximal end side along the longitudinal axis L is a polygon, such as a rectangle, as illustrated in FIG. 5A. When the projected shape of the resection portion 82 is an approximate rectangle, it is preferable that the size of the rectangle is about 4 millimeters (mm)×5 mm. In some other embodiments, the projected shape of the resection portion 82 viewed from the distal end side to the proximal end side along the longitudinal axis L is an ellipse as illustrated in FIG. 5B.

Furthermore, the projected shape of the resection portion 82 may be a rectangle with curved corners as an approximate polygon, or a shape of a racetrack at an athletics stadium as an approximate ellipse. Therefore, the projected shape of the resection portion 82 has an appropriate shape, such as a polygon, an approximate polygon, an ellipse, or an approximate ellipse.

The resection portion 82 includes a columnar portion 86 and a protruding portion 87 that protrudes from the columnar portion 86 to the distal end side along the longitudinal axis L. The columnar portion 86 is in the form of a column, such as a polygonal column or an elliptical column. For example, the columnar portion 86 has an appropriate shape, such as a triangular prism, a quadrangular prism, a pentagonal prism, or a hexagonal prism, or a shape similar to the above-described shapes. The columnar portion 86 needs not always have specific corners. The columnar portion 86 includes side surfaces 86a. The side surfaces 86a form an outer peripheral surface of the columnar portion 86 and form a part of the side surfaces 83 of the treatment unit 74. The side surfaces 86a are formed approximately parallel to the longitudinal axis L.

The protruding portion 87 protrudes to the distal end side from the columnar portion 86 along the longitudinal axis L. The protruding portion 87 extends along the longitudinal axis L and is in the form of a frustum or an approximate frustum. The protruding portion 87 is constructed as an enlarging portion that enlarges, from the distal end to the proximal end along the longitudinal axis L, a cross-sectional area of a cross-section of the bone perpendicular to the longitudinal axis L. In this example, in particular, the protruding portion 87 is constructed as a diameter enlarging portion that enlarges an outer diameter from the distal end to the proximal end. The protruding portion 87 includes inclined surfaces 87a that form side surfaces of a frustum or an approximate frustum, and a distal end surface 87b that forms a distal end of the protruding portion 87. The inclined surfaces 87a and the distal end surface 87b form an outer surface of the protruding portion 87. The inclined surfaces 87a form a part of the side surfaces 83 of the treatment unit 74. The inclined surfaces 87a are oriented so as to approach the longitudinal axis L from the proximal end to the distal end. Therefore, the inclined surfaces 87a are inclined with respect to the longitudinal axis L. A projected shape of the protruding portion 87 viewed from the distal end side to the proximal end side along the longitudinal axis L is included in a range of a projected shape of the columnar portion 86. Meanwhile, the outer surface of the protruding portion 87 may be formed by only the inclined surfaces 87a.

The treatment unit 74 includes a discharge unit 84 that discharges resection debris (debris) of the bone resected by the resection portion 82 to the proximal end side relative to the resection portion 82 along the longitudinal axis L. A part of the discharge unit 84 is provided in the resection portion 82. The discharge unit 84 includes a recess 92 that is formed on an outer peripheral surface of the resection portion 82, and a shaft portion 94. The shaft portion 94 extends to the proximal end side relative to the columnar portion 86 of the resection portion 82 along the longitudinal axis L. The shaft portion 94 is provided between the distal end of the probe main body 72 and a proximal end of the columnar portion 86.

A cross-sectional area of a cross-section of the shaft portion 94 perpendicular to the longitudinal axis L is reduced from the distal end side to the proximal end side. Therefore, the shaft portion 94 is reduced from the distal end side to the proximal end side. When the treatment unit 74 is viewed from the distal end side to the proximal end side along the longitudinal axis L, a projected shape of the shaft portion 94 is not observable because it is hidden behind the projected shape of the columnar portion 86.

The recess 92 is formed on the side surfaces 86a of the columnar portion 86 and the inclined surfaces 87a of the protruding portion 87. For example, the recess 92 is a groove that is extended in a spiral manner around the longitudinal axis L. Therefore, in a portion where the recess 92 is provided, an area that comes in contact with a bone when the bone is resected is reduced. Further, the recess 92 serves as a discharge path through which bone resection debris that occurs when the bone is resected moves toward the proximal end side.

As described above, the projected shapes of the protruding portion 87 and the shaft portion 94 viewed from the distal end side to the proximal end side along the longitudinal axis L are included in the range of the projected shape of the columnar portion 86. Therefore, the columnar portion 86 serves as a maximum outer contour portion of the resection portion 82 and the treatment unit 74, and defines the projected shapes of the resection portion 82 and the treatment unit 74 when viewed from the distal end side to the proximal end side along the longitudinal axis L. Therefore, when the recess 92 is provided on the side surfaces 86a of the columnar portion 86, the side surfaces 86a are constructed so as not to change the desired projected shape of the columnar portion 86.

A guide unit 95 that extends from the distal end surface 87b of the protruding portion 87 to the distal end side along the longitudinal axis L is provided on a distal end side of the treatment unit 74. The guide unit 95 is provided in a continuous manner on a distal end side of the protruding portion 87. In other words, the guide unit 95 protrudes from the distal end of the protruding portion 87 of the treatment unit 74 to the distal end side. The treatment unit 74 and the guide unit 95 may be constructed in an integrated manner. The guide unit 95 includes an extended portion 96 that extends along the longitudinal axis L, and a distal end forming portion 97 that is provided on a distal end side of the extended portion 96. The distal end forming portion 97 forms a distal end of the guide unit 95.

The extended portion 96 is in the form of an approximate column that extends from the distal end surface 87b of the resection portion 82 to the distal end side along the longitudinal axis L. Therefore, a cross-sectional shape of the extended portion 96 perpendicular to the longitudinal axis L is an approximate ellipse. A projected shape of the extended portion 96 viewed from the distal end side to the proximal end side along the longitudinal axis L is included in a range of the projected shapes of the distal end surface 87b of the protruding portion 87 and the columnar portion 86. The cross-sectional shape of the extended portion 96 perpendicular to the longitudinal axis L may be an approximate polygon, an approximate ellipse, or an approximate star. A cross-section of the extended portion 96 perpendicular to the longitudinal axis L has an approximately constant shape or an approximately constant area from the distal end to the proximal end.

The distal end forming portion 97 is in the form of a hemisphere that protrudes from a distal end of the extended portion 96 to the distal end side. Therefore, an outer surface of the distal end forming portion 97 is formed by a curved surface. A cross-section of a proximal end of the distal end forming portion 97 perpendicular to the longitudinal axis L is approximately the same as a cross-section of the distal end of the extended portion 96. The cross-section of the distal end forming portion 97 perpendicular to the longitudinal axis L is reduced from the proximal end side to the distal end side. Therefore, a diameter of the distal end forming portion 97 is reduced from the proximal end side to the distal end side. Therefore, a projected shape of the distal end forming portion 97 viewed from the distal end side to the proximal end side along the longitudinal axis L is included in a range of the projected shape of the resection portion 82. Therefore, the extended portion 96 defines a maximum outer contour of the guide unit 95.

The distal end forming portion 97 and the extended portion 96 may be constructed in an integrated manner. Further, the cross-sectional shape of the proximal end of the distal end forming portion 97 may be approximately the same as the cross-sectional shape of the extended portion 96 or may be different from the cross-sectional shape of the extended portion 96.

The extended portion 96 defines the maximum outer contour of the guide unit 95. Further, the maximum outer contour of the guide unit 95 has a shape that is insertable into a small hole that is made in a bone to be treated. Therefore, a maximum outer diameter of the guide unit 95 is set to be smaller than an outer diameter of a small hole that is made in a bone. For example, when an outer diameter of a drill for making a small hole is 4.0 mm, the outer diameter of the extended portion 96 is set to 3.8 mm. Furthermore, when the outer diameter of the drill for making a small hole is 4.5 mm, the outer diameter of the extended portion 96 is set to 4.3 mm.

The distal end forming portion 97 may include a portion with an outer diameter larger than the outer diameter of the extended portion 96. In this case, a maximum outer contour of the distal end forming portion 97 becomes larger than an outer contour of the extended portion 96. Therefore, the maximum outer contour of the distal end forming portion 97 serves as the maximum outer contour of the guide unit 95. The maximum outer contour of the guide unit 95 has a shape that is insertable into a small hole that is made in a bone to be treated.

Therefore, the maximum outer contour of the distal end forming portion 97 is set to be smaller than an outer contour of the small hole.

Here, for example, a concave hole (large hole) with a desired shape includes an opening edge portion that has the same shape and the same size as those of the projected shape of the resection portion 82 of the treatment unit 74 viewed from the distal end side to the proximal end side along the longitudinal axis L, and is recessed toward an inner side in a straight manner while maintaining the same shape as the opening edge portion.

Therefore, one example of the desired large hole is a rectangle with an appropriate depth.

To make a large hole with a desired shape, it is necessary that a projected shape of the maximum outer contour portion of the resection portion 82 of the treatment unit 74 viewed from the distal end side to the proximal end side along the longitudinal axis L has the same shape as the opening edge portion of the desired large hole. The columnar portion 86 of the resection portion 82 of the treatment unit 74 has the same shape as the shape of the opening edge portion of the desired large hole. Therefore, it is possible to make the large hole with the desired opening edge portion with the aid of the columnar portion 86 of the resection portion 82 of the treatment unit 74 of the ultrasonic probe 66.

In contrast, from the viewpoint of reducing friction between the bone and the resection portion 82 of the treatment unit 74 and from the viewpoint of discharging resection debris generated from the bone, it is preferable to reduce a length of the maximum outer contour portion of the resection portion 82 in a direction along the longitudinal axis L (in a direction of ultrasonic vibration). Therefore, it may be preferable that the columnar portion 86 serving as the maximum outer contour portion has a cross-sectional area that is gradually reduced from the distal end side to the proximal end side, instead of having a constant shape and a constant cross-sectional area.

It is preferable to move the ultrasonic probe 66 in a straight manner along the longitudinal axis L, and make a large hole in a straight manner along the longitudinal axis L by the resection portion 82. Therefore, to prevent wobble of the resection portion 82 and make the large hole in a straight manner, an outer contour of the columnar portion 86 from the distal end to the proximal end needs to have a certain length parallel to the longitudinal axis L.

Furthermore, the treatment unit 74 resects a bone while ultrasonic vibration at appropriate amplitude is being transmitted to the ultrasonic probe 66. Therefore, the columnar portion 86 of the resection portion 82 of the treatment unit 74 needs to have appropriate strength. If the cross-sectional area of the columnar portion 86 is gradually reduced from the distal end side to the proximal end side, in some cases, it may be difficult to construct the treatment unit 74 with certain strength that is necessary to cause the treatment unit 74 to resect a bone while transmitting ultrasonic vibration at appropriate amplitude to the ultrasonic probe 66, depending on a diminution rate of the cross-sectional area or the like.

In the columnar portion 86 of the resection portion 82 of the ultrasonic probe 66, a region constituting the maximum outer contour portion is maintained from the distal end to the proximal end and has a certain length along the longitudinal axis L. Further, a cross-section of the columnar portion 86 of the resection portion 82 perpendicular to the longitudinal axis L is constant or approximately constant from the distal end to the proximal end of the columnar portion 86. In this manner, by providing the columnar portion 86 in the resection portion 82 of the treatment unit 74, it is possible to maintain the strength of the treatment unit 74 when the ultrasonic probe 66 is moved in a straight manner toward the distal end side along the longitudinal axis L, and make a large hole in a straight manner while maintaining the same shape as a maximum outer contour portion of the columnar portion 86 at the time of resecting a bone.

Meanwhile, when the columnar portion 86 has an appropriate length along the longitudinal axis L and the recess 92 of the discharge unit 84 is not provided, friction between the bone and the outer peripheral surface of the columnar portion 86 increases. The maximum outer contour portion of the columnar portion 86 is maintained from the distal end to the proximal end along the longitudinal axis L, and therefore, an outer contour of the region perpendicular to the longitudinal axis L is constant at any position from the distal end to the proximal end. Therefore, if the recess 92 of the discharge unit 84 is not provided, resection debris generated from a bone resected by the distal end of the columnar portion 86 is caught between the bone and the outer peripheral surface of the columnar portion 86 and is less likely to be discharged.

The recess 92 of the discharge unit 84 of the ultrasonic probe 66 is formed in the columnar portion 86. The recess 92 of the discharge unit 84 does not change a projected shape of the maximum outer contour portion of the columnar portion 86 when the treatment unit 74 is viewed from the distal end side to the proximal end side along the longitudinal axis L. Further, the recess 92 is provided in a continuous manner from the distal end to the proximal end of the columnar portion 86. Therefore, once resection debris enters the recess 92, the resection debris moves along the recess 92 toward the proximal end side relative to the treatment unit 74 along with forward movement of the ultrasonic probe 66 along the longitudinal axis L. Therefore, the treatment unit 74 of the ultrasonic probe 66 solves problems with the friction between the bone and the resection portion 82, discharge of resection debris generated when the bone is resected by the resection portion 82, and the strength of the resection portion 82.

A cross-sectional area of the shaft portion 94 of the discharge unit 84 is reduced from the distal end side to the proximal end side. Further, in the ultrasonic probe 66, a proximal end of the shaft portion 94 and the distal end of the probe main body 72 form a constricted part. Therefore, the shaft portion 94 of the discharge unit 84 is able to form a space for discharging resection debris between an inner wall of the concave hole in the bone and the shaft portion 94.

Next, functions and effects of the treatment system 10 will be described. The treatment system 10 is used for, for example, treatment to make a bone hole (a through hole or a concave hole) for fixing a transplant ligament in a femur and/or a tibia in ACL reconstruction.

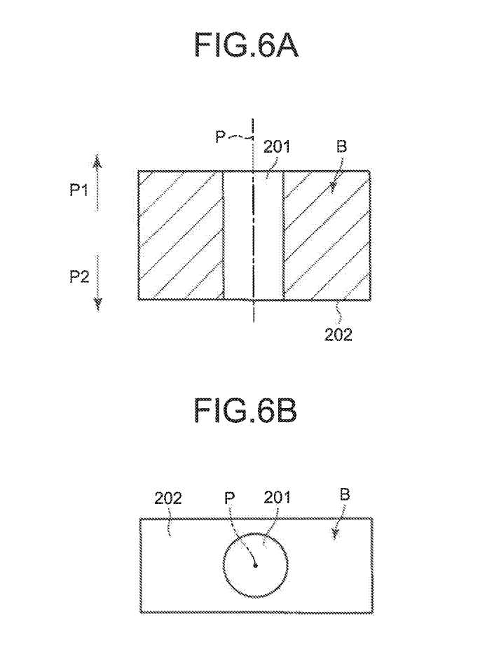

In this treatment, a small hole (through hole) for inserting a fixator to be connected to the transplant ligament is made in a bone serving as a treatment target, by using a drill or the like that is inserted in the joint space 136 of the knee joint 100 from the second portal 104, for example. FIG. 6A and FIG. 6B are diagrams illustrating a small hole 201 that is made in a bone B serving as a treatment target. As illustrated in FIG. 6A and FIG. 6B, the small hole 201 has a central axis P. FIG. 6A illustrates a cross-section of the small hole 201 including the central axis P. FIG. 6B illustrates the small hole 201 viewed from one side in a direction along the central axis P.

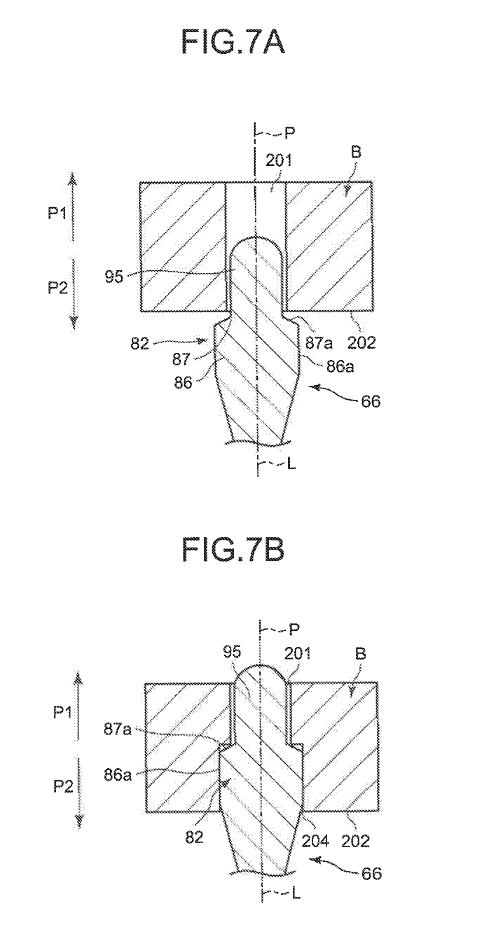

The treatment unit 74 of the ultrasonic treatment tool 52 is inserted in the joint space 136 inside the knee joint 100 and the switch 36 is pressed. Accordingly, the controller 34 outputs electrical energy and the transducer 56b serving as an ultrasonic transducer generates ultrasonic vibration. Then, longitudinal vibration occurs in the ultrasonic probe 66 in a direction parallel to the central axis C, and the ultrasonic vibration is transmitted to the treatment unit 74 provided in the ultrasonic probe 66. In this state, by moving the ultrasonic probe 66 along the central axis P of the small hole 201, a portion of the bone B in contact with the resection portion 82 of the treatment unit 74 is resected, so that a large hole (concave hole) for inserting the transplant ligament is made along the central axis P of the small hole 201. In this case, a cross-sectional shape of a large hole 204 includes a cross-sectional shape of the small hole 201. Further, the treatment unit 74 is an ultrasonic treatment unit that treats a treatment target, such as a bone, by the transmitted ultrasonic vibration. FIG. 7A and FIG. 7B are diagrams illustrating a state in which the large hole 204 is made in the bone B in which the small hole 201 has been made. FIG. 7A and FIG. 7B illustrate cross-sections of the small hole 201 including the central axis P.

As illustrated in FIG. 6A and FIG. 6B, the small hole 201 includes the central axis P. FIG. 6A illustrates the cross-section of the small hole 201 including the central axis P. FIG. 6B illustrates the small hole 201 viewed from one side in a direction along the central axis P. The bone B has a treatment surface 202 to be subjected to treatment using ultrasonic vibration. The small hole 201 is extended along the central axis P. The small hole 201 is a circular hole that is made by a drill or the like and has an approximately circular cross-section. An approximately circular opening is formed in the treatment surface 202 of the bone B. Here, a side from the treatment surface 202 of the bone B to an inner side of the bone B in a direction along the central axis P will be referred to as an inner side (an arrow P1 side in FIG. 6A), and a side opposite to the inner side will be referred to as a front side (an arrow P2 side in FIG. 6A).

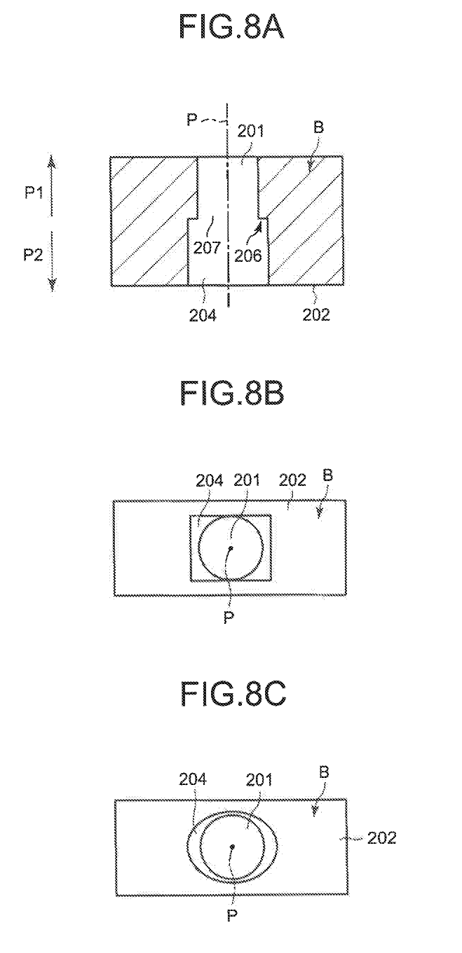

FIG. 8A to FIG. 8C are diagrams illustrating the large hole 204 made in the bone B. FIG. 8A illustrates a cross-section including the central axis P. FIG. 8B and FIG. 8C illustrate the treatment surface 202 of the bone B viewed from the front side to the inner side along the central axis P. As illustrated in FIG. 7A to FIG. 8C, the large hole 204 is made in a continuous manner on the front side of the small hole 201. It is preferable that a central axis of the large hole 204 coincides with the central axis P of the small hole 201. The large hole 204 is extended from the treatment surface 202 of the bone B to the inner side along the central axis P, and the small hole 201 is extended from an inner end portion of the large hole 204 to the inner side along the central axis P. A cross-sectional shape the small hole 201 is included in the cross-sectional shape of the large hole 204.

Therefore, an opening 207 is formed by the small hole 201 in a bottom surface 206 of the large hole 204. Further, a projected shape of the large hole 204 viewed from the front side to the inner side along the central axis P includes a projected shape of the small hole 201.

It is preferable that the treatment unit 74 moves along the longitudinal axis L relative to the bone B that is a treatment target. In this case, a direction along the longitudinal axis L corresponds to a resection direction of the treatment unit 74 and the resection portion 82. The cross-sectional shape of the large hole 204 is defined by a projected shape of the treatment unit 74 viewed from the distal end side to the proximal end side along the longitudinal axis L. Therefore, the cross-sectional shape of the large hole 204 is a polygon, such as an approximate rectangle, as illustrated in FIG. 8B or an ellipse as illustrated in FIG. 8C depending on the projected shape of the treatment unit 74.

The guide unit 95 is extended along the longitudinal axis L on a distal end side of the resection portion 82.

The maximum outer contour of the guide unit 95 has a shape that is insertable into the small hole 201 along the longitudinal axis L. As illustrated in FIG. 7A, when the large hole 204 is to be made in the bone B, the guide unit 95 is inserted into the small hole 201 from the front side. By inserting the guide unit 95 into the small hole 201 up to the proximal end of the guide unit 95, a position of the treatment unit 74 relative to the treatment surface 202 of the bone B is adjusted to an appropriate position.

Further, while the guide unit 95 is inserted in the small hole 201, the longitudinal axis L of the guide unit 95 approximately coincides with the central axis P of the small hole 201. In this case, the longitudinal axis L of the guide unit 95 and the central axis P of the small hole 201 become approximately parallel to each other. Furthermore, by moving the treatment unit 74 along the longitudinal axis L, the treatment unit 74 and the resection portion 82 move along the central axis P relative to the bone B. By moving the resection portion 82 along the central axis P of the small hole 201, the resection direction of the resection portion 82 approximately coincides with the central axis P of the small hole 201. In this case, the resection direction of the resection portion 82 and the central axis P of the small hole 201 become approximately parallel to each other. Because the resection direction of the resection portion 82 coincides or approximately coincides with the central axis P of the small hole 201, the large hole 204 is made along the central axis P of the small hole 201. Meanwhile, the large hole 204 may be made such that the central axis thereof becomes approximately parallel to the central axis P of the small hole 201.

As described above, by providing the guide unit 95 on the distal end side of the resection portion 82, the position of the treatment unit 74 relative to the small hole 201 and the resection direction (longitudinal axis L) of the treatment unit 74 relative to the central axis P of the small hole 201 are adjusted. In other words, the treatment unit 74 is guided by the guide unit 95 so as to move along the longitudinal axis L during resection operation. Therefore, it is possible to prevent a situation in which the central axis of the large hole 204 is inclined with respect to the central axis P of the small hole 201. By making the large hole 204 with respect to the central axis P of the small hole 201, it is possible to make bone holes (the small hole 201 and the large hole 204) in which a transplant ligament (tendon transplant) and a fixator connected to the tendon transplant are appropriately insertable.

Meanwhile, it is preferable that operation on the switch 36 for causing the transducer 56b to generate ultrasonic vibration is performed while the guide unit 95 is inserted in the small hole 201 as illustrated in FIG. 7A.

In some embodiments, a groove is formed on an outer surface of the guide unit 95. In this case, even when the outer surface of the guide unit 95 comes in contact with an inner peripheral surface of the small hole 201 while the guide unit 95 is inserted into a through hole, a contact area between the outer surface of the guide unit 95 and the inner peripheral surface of the small hole 201 is reduced. Therefore, it is possible to prevent a contact portion between the guide unit 95 and the small hole 201 from being unnecessarily resected.

Furthermore, the recess 92 of the discharge unit 84 is formed on each of the protruding portion 87 and the columnar portion 86 of the treatment unit 74. Resection debris of the bone B resected by the resection portion 82 is disposed in the recess 92. Then, the resection debris of the bone B is discharged to a proximal end side of the resection portion 82 through the recess 92. Moreover, the surface area of the resection portion 82 is increased because of the recess 92, so that heat dissipation can be improved.

Furthermore, the protruding portion 87 includes the inclined surfaces 87a. Therefore, the resection debris of the bone B resected by the inclined surfaces 87a moves to an outer peripheral side along the inclined surfaces 87a and is discharged to the proximal end side through the recess 92 formed in the columnar portion 86.

Therefore, it is possible to effectively discharge the resection debris of the bone B generated by the resection portion 82 to the proximal end side relative to the resection portion 82.

Moreover, the shaft portion 94 is not observable when the proximal end of the columnar portion 86 is viewed from the distal end side to the proximal end side along the longitudinal axis L. Therefore, when the large hole 204 is to be made, a space is formed between an outer peripheral surface of the shaft portion 94 and an inner wall of the large hole 204. Therefore, on the proximal end side of the columnar portion 86, the resection debris of the bone B is discharged to the proximal end side along the longitudinal axis L through the space between the shaft portion 94 and the inner wall of the large hole 204.

As described above, the discharge unit 84 (the recess 92 and the shaft portion 94) is a discharge mechanism that discharges the resection debris of the bone B resected by the treatment unit 74 to the proximal end side relative to the resection portion 82. By effectively discharging the resection debris of the bone B to the proximal end side relative to the resection portion 82, it is possible to increase a resection speed for resecting the bone B.

Furthermore, it may be possible to provide only one of the shaft portion 94 and the recess 92 or both of them as the discharge unit 84. Moreover, when the recess 92 is provided as the discharge unit 84, the recess 92 may be provided on only one of the columnar portion 86 and the protruding portion 87 or on both of them.

Meanwhile, the recess 92 may be a crosshatched groove or the like. Furthermore, the recess 92 may be formed by blasting, such as sandblasting.

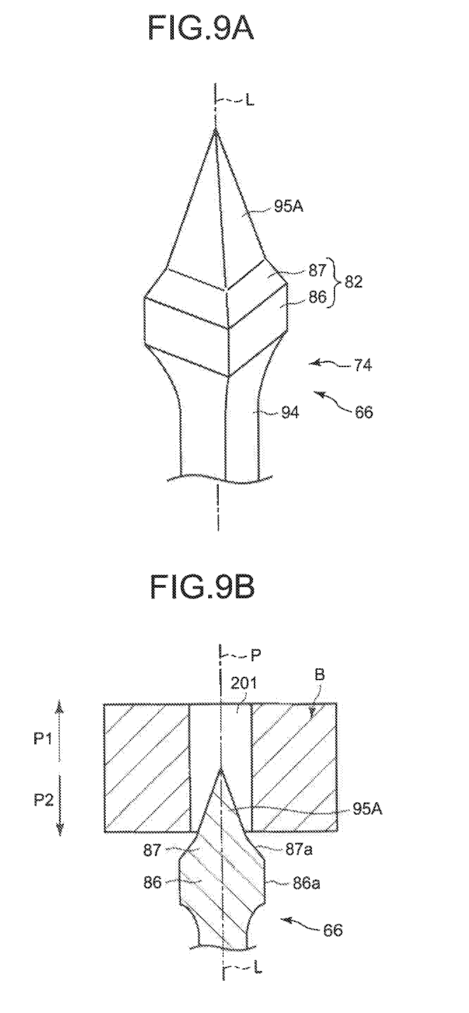

FIG. 9A is a diagram illustrating a configuration of the treatment unit 74 of the ultrasonic probe 66. As illustrated in FIG. 9A, a guide unit 95A can be disposed on the distal end side of the treatment unit 74.

A cross-sectional area of the guide unit 95A perpendicular to the longitudinal axis L is reduced toward the distal end side. The guide unit 95A is in the form of a quadrangular pyramid that is extended toward the distal end of the treatment unit 74, i.e., toward the distal end side, along the longitudinal axis L. A cross-sectional shape of the guide unit 95A may be a polygon, a circle, a star, or the like. The cross-sectional shape of a proximal end of the guide unit 95A perpendicular to the longitudinal axis L is included in the cross-sectional shape of the small hole 201. Therefore, it is possible to insert the guide unit 95A in the small hole 201 that is made in a bone to be treated.

FIG. 9B is a diagram illustrating a state in which the guide unit 95A is inserted in the small hole 201 made in the bone B. Even in the second embodiment, by inserting the guide unit 95A in the small hole 201, the position of the treatment unit 74 relative to the treatment surface 202 of the bone B is adjusted to an appropriate position. Further, by moving the treatment unit 74 relative to the bone B along the longitudinal axis L while the longitudinal axis L and the central axis P of the small hole 201 coincide or approximately coincide with each other, the large hole 204 is made along the central axis P of the small hole 201.

In the second embodiment, the cross-sectional area of the guide unit 95A perpendicular to the longitudinal axis L is reduced from the proximal end side to the distal end side. Therefore, as compared to a case in which the cross-sectional area perpendicular to the longitudinal axis L is constant, it is possible to improve insertion capability with respect to the small hole 201. Furthermore, while the guide unit 95A is inserted in the small hole 201, a space between an outer surface of the guide unit 95A and an inner surface of the small hole 201 is increased toward the distal end side. Therefore, it is possible to prevent a distal end portion of the guide unit 95A from coming in contact with the inner wall of the small hole 201. By preventing contact with the inner wall of the small hole 201, it is possible to prevent the inner wall of the small hole 201 from being resected.

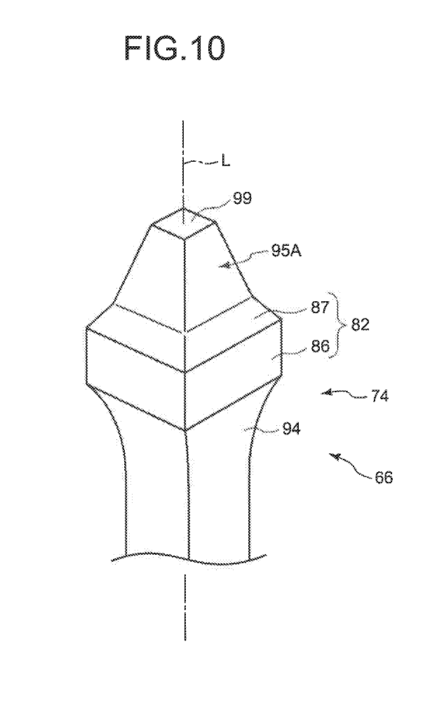

As illustrated in FIG. 10, it is also possible to dispose a flat surface portion 99 oriented toward the distal end side on a distal end of the guide unit 95A. In the modification, even when the distal end portion of the guide unit 95A comes in contact with unintended tissue, a force applied to a portion of the guide unit 95A in contact with the bone B is likely to be distributed. Therefore, the guide unit 95A is less likely to be damaged even when the distal end portion comes in contact with unintended tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic probe comprising:
a probe main body configured to receive ultrasonic vibration generated by an ultrasonic transducer, the probe body including a distal end side and a proximal end side;
a treatment unit disposed on the distal end side of the probe main body along a longitudinal axis, the treatment unit being configured to form a hole in a bone to be treated; the treatment unit including:
a resection portion configured to resect the bone when the treatment unit moves along the longitudinal axis while the ultrasonic vibration is being transmitted to the probe main body; and
a discharge mechanism configured to discharge debris of the bone resected by the resection portion such that the debris is discharged proximally of the resection portion; and
a guide unit that protrudes from a distal end of the treatment unit along a resection direction of the resection portion, the guide unit being configured to be inserted into the hole formed by the treatment unit.

2. The ultrasonic probe according to claim 1, wherein the guide unit is configured to move relative to the bone along the hole in the bone along a resection direction of the resection portion such that the resection direction of the resection portion remains parallel to a central axis of the hole.

3. The ultrasonic probe according to claim 1, wherein the resection portion includes an enlarging portion that is configured to enlarge a cross-sectional area of a cross-section of the bone perpendicular to the longitudinal axis.

4. The ultrasonic probe according to claim 3, wherein the guide unit extends continuously to a distal end side of the enlarging portion.

5. The ultrasonic probe according to claim 1, wherein the treatment unit includes a first projection shape that, when viewed along the longitudinal axis, has any one of the following shapes: polygon shape and round shape.

6. The ultrasonic probe according to claim 1, wherein a distal end portion of the guide unit is in a form of a hemisphere.

7. The ultrasonic probe according to claim 5, wherein the guide unit includes a second projection shape that, when viewed along the longitudinal axis, has any one of the following shapes: polygon shape and round shape.

8. The ultrasonic probe according to claim 7, wherein an outline of the first projection shape encompasses an outline of the second projection shape.

9. The ultrasonic probe according to claim 7, wherein an outline of the first projection shape coincides with an outline of the second projection shape.

10. A treatment unit for an ultrasonic probe configured to form a hole in a bone to be treated, comprising:
a guide unit provided on a distal end of the treatment unit, the guide unit being configured to be inserted into the hole formed by the treatment unit; and
a resection portion that extends proximally from the guide unit along a longitudinal axis, the resection portion being configured to resect the bone along the longitudinal axis.

11. The treatment unit according to claim 10, further comprising a discharge mechanism configured to discharge debris of the bone resected by the resection portion such that the debris is discharged proximally of the resection portion.

12. The treatment unit according to claim 10, wherein the guide unit is configured to guide the treatment unit to move relative to the bone along the resection direction such that the resection direction remains parallel to a central axis of the hole.

13. The treatment unit according to claim 10, wherein the resection portion includes a first projection shape that, when viewed along the longitudinal axis, has any one of the following shapes: polygon shape and round shape.

14. The treatment unit according to claim 13, wherein the guide unit includes a second projection shape that, when viewed along the longitudinal axis, has any one of the following shapes: polygon shape and round shape.

15. The treatment unit according to claim 14, wherein an outline of the first projection shape encompasses an outline of the second projection shape.

16. The treatment unit according to claim 14, wherein an outline of the first projection shape coincides with an outline of the second projection shape.
